(19) United States
(12) Patent Application Publication
(10) Pub. No.: US 2026/0345900 A1
(43) Pub. Date: Nov. 5, 2026

(54) AUTOMATIC FLOW STOP FOR INFUSION SET

(71) Applicant: CareFusion 303, Inc., San Diego, CA (US)

(72) Inventor: Manuel Anaya PEREA, San Diego, CA (US)

(21) Appl. No.: 19/195,558

(22) Filed: Apr. 30, 2025

(51) Int. Cl. A61M 5/168; A61M 39/28

(52) U.S. Cl. CPC A61M 5/16813; A61M 39/28; A61M 2205/3337

(57) ABSTRACT

The intravenous (IV) flow stop described herein automatically and fully occludes flow in an IV line coupled to an infusion pump when the infusion pump is open and facilitates flow when the infusion pump is closed. The flow stop includes a housing with an opening that receives the IV line, a slider coupled to the housing to transition the flow stop between occluding and flow configurations, and a biasing member coupled to the housing to bias the flow stop towards the occluding configuration. The slider transitions the flow stop between the occluding and flow configurations via a clamping aperture with an occluding region and a flow region. Specifically, the occluding region aligns with the opening in the housing to receive the IV line when the flow stop is in the occluding configuration, and the flow region aligns with the opening when the flow stop is in the flow configuration.

### TECHNICAL FIELD

The invention is generally related to the field of intravenous (“IV”) infusion devices such as infusion pumps and the associated flexible IV tubing and flow stop devices and, more particularly, to devices used to prevent free flow of a fluid in an IV tube when the infusion pump is disengaged from the IV tube.

### BACKGROUND

It is a common practice to deliver fluids such as medications to a patient intravenously by means of a pumping device such as a large volume pump. Such pumps are useful because they can deliver the medication in a highly controlled fashion, and because they do so without coming in contact with the medication. The fluid is moved through a flexible IV tube by pressing one or more pumping fingers against the outer surface of the tube sufficiently to force fluid downstream through the tube into the patient.

It is common for the pumping mechanism of a finger pump to be disposed in a housing with a hinged door. The tube through which the fluid is to be moved is placed in contact with the pumping mechanism inside the door, with the upstream and downstream ends of the tubing typically extending out the top and bottom, respectively, of the door opening. As the door is shut over the tube, the door provides a backing surface against which the pumping fingers can occlude the tube.

This arrangement of the IV tube relative to the pumping mechanism requires that there be some means for preventing flow in the tube when the door is open. Otherwise, during the process of mounting or removing the tube from the pumping mechanism, an unwanted flow of fluid could occur in the IV tube. This could result in the uncontrolled infusion of medication into the patient under the influence of the static head pressure in the tube.

### SUMMARY

Devices for preventing unwanted flow in an IV tube include manual clamps separate from the infusion pump as well as occluding devices mounted on the door of the pump. Manual clamp devices require some manipulation skill on the part of the attending technician, and there is the chance that the technician will forget to properly time the occlusion of the tube relative to the opening of the door on the pumping device.

The door-mounted occluding devices can require that the door push the devices relative to the tube to occlude the tube when the door is opened and open the tube when the door is closed. However, these door-mounted occluding devices require that the door be fully opened or fully closed at all times and can facilitate inadvertent flow through the tube if the door is left half open, if the door is closed too slowly, etc.

Therefore, those skilled in the art have recognized a need for an apparatus that will automatically and fully occlude the IV tube when the door on the pumping device begins to open and then permit flow through the IV tube when the door is fully closed. This apparatus limits the need for an attending technician or other caregiver to monitor flow through the IV tube when operating the pumping device. Accordingly, some implementations of the present disclosure are directed to an IV flow stop having an occluding configuration and a flow configuration, the flow stop comprising: a housing comprising an opening configured to receive an IV tube; a slider slidably coupled to the housing to transition the flow stop between the occluding configuration and the flow configuration, wherein the slider comprises a clamping aperture that has an occlusion slot, has a flow opening, and is configured to receive the IV tube; and a biasing member coupled to the slider and the housing to bias movement of the slider along the housing, such that the flow stop is biased towards the occluding configuration, wherein, when the flow stop is in the occluding configuration, the occlusion slot overlaps the opening in the housing to occlude the IV tube, wherein, when the flow stop is in the flow configuration, the flow opening overlaps the opening in the housing to permit flow through the IV tube.

In some implementations, the flow stop is configured to be coupled to an interior of an infusion pump. Optionally, the IV flow stop is configured to enter the flow configuration when a door of the infusion pump is closed because the closed door slides the slider until the flow opening overlaps the opening in the housing. Optionally, the IV flow stop is configured to enter the occluding configuration automatically when a door of the infusion pump is begins to open because the biasing member slides the slider until the occlusion slot overlaps the opening in the housing.

In some implementations, the IV tube is transverse to the housing and the slider slides parallel to the housing.

In some implementations, the flow stop further comprises a port extending from the opening of the housing transverse to the housing, wherein the port is configured to receive the IV tube.

In some implementations, the occlusion slot is narrower than the flow opening, such that the occlusion slot is configured to pinch the IV tube to occlude the IV tube.

In some implementations, the biasing member comprises a torsion spring. Optionally, a first end of the torsion spring is coupled to a distal portion of the housing and a second end of the torsion spring is coupled to a distal portion of the slider. Optionally, in a pretensioned position, the torsion spring is configured to urge the distal portion of the slider away from the distal portion of the housing, and, in a loaded position, the torsion spring allows the distal portion of the housing and the distal portion of the slider to slide towards each other. Optionally, the flow stop is in the occluding configuration when the torsion spring is in the pretensioned position, and the flow stop is in the flow configuration when the torsion spring is in the loaded position.

Some implementations of the present disclosure are directed to an IV flow stop having an occluding configuration and a flow configuration, the flow stop comprising: a housing configured to couple with an IV set; a slider slidably coupled to the housing and comprising a clamping mechanism that is configured to receive the IV set and slide relative to the IV set when the slider slides relative to the housing; and a biasing member coupled to the slider and the housing, wherein the biasing member biases the flow stop towards the occluding configuration, wherein the clamping mechanism occludes flow through the IV set when the flow stop is in the occluding configuration and permits flow through the IV set when the flow stop is in the flow configuration.

In some implementations, the clamping mechanism comprises: an occluding region configured to occlude the IV set by engaging the IV set when the flow stop is in the occluding configuration; and a flow region configured to permit flow through the IV set by engaging the IV set when the flow stop is in the flow configuration.

In some implementations, the biasing member comprises a torsion spring. Optionally, the flow stop is in the occluding configuration when the torsion spring is in a pretensioned position, and the flow stop is in the flow configuration when the torsion spring is in a loaded position.

In some implementations, the biasing member comprises a compression spring.

Other implementations of the present disclosure are directed to an IV flow stop having an occluding configuration and a flow configuration, the flow stop comprising: a housing configured to couple with an IV set; a slider slidably coupled to the housing, comprising a clamping mechanism, and configured to couple with the IV set; and a biasing member protruding from a distal portion of the slider, wherein the biasing member is configured to bias the flow stop towards the occluding configuration, wherein, to enter the occluding configuration, the biasing member is configured to expand to slide the slider proximally relative to the housing, such that the clamping mechanism occludes flow through the IV set, wherein, to enter the flow configuration, the biasing member is configured to contract to slide the housing distally relative to the slider, such that the clamping mechanism permits flow through the IV set.

In some implementations, the housing comprises a port configured to connect the housing to the IV set.

In some implementations, the clamping mechanism comprises an occluding region configured to occlude the IV set and a flow region configured to permit flow through the IV set, wherein the clamping mechanism is configured to move relative to the IV set when the slider slides relative to the housing, such that the occluding region is configured to couple with the IV set when the flow stop is in the occluding configuration and the flow region is configured to couple with the IV set when the flow stop is in the flow configuration.

In some implementations, the biasing member comprises a compression spring.

Additional features and advantages of the subject technology will be set forth in the description below, and in part will be apparent from the description, or may be learned by practice of the subject technology. The advantages of the subject technology will be realized and attained by the structure particularly pointed out in the written description and implementations hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative implementations of the inventions are described below with reference to the drawings. The illustrated implementations are intended to illustrate, but not to limit, the inventions. The drawings contain the following figures:

FIG. 1 illustrates an infusion pump loaded with an IV set configured for use, in accordance with some implementations disclosed herein.

FIG. 2 illustrates a perspective view of the infusion pump shown in FIG. 1 with its door opened, revealing an example IV flow stop coupled thereto, in accordance with some implementations disclosed herein.

FIG. 3A illustrates a perspective top view of a first implementation of the IV flow stop in the occluding configuration, in accordance with some implementations disclosed herein.

FIG. 3B illustrates a perspective top view of the first implementation of the IV flow stop in the flow configuration, in accordance with some implementations disclosed herein.

FIG. 3C illustrates a perspective bottom view of the first implementation of the IV flow stop with the housing and the slider separated, in accordance with some implementations disclosed herein.

FIG. 3D illustrates the IV flow stop of FIG. 3C with a port attached to the opening, in accordance with some implementations disclosed herein.

FIG. 4 illustrates a perspective top view of a second implementation of the IV flow stop in the occluding configuration, in accordance with some implementations disclosed herein.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various implementations, it will be appreciated that the description is illustrative only and should not be construed in any way as limiting. Additionally, it is contemplated that although particular implementations of the present disclosure may be disclosed or shown in the context of an IV set, such implementations can be used in other fluid conveyance systems. Furthermore, various applications of such implementations and modifications thereto, which may occur to those who are skilled in the art, are also encompassed by the general concepts described herein.

It may be desirable to occlude fluid flow through a section of an infusion line running through an infusion pump while the door of an infusion pump is open If the door contributes to the pump's ability to occlude the infusion line, then another device may be required to prevent unwanted flow in the infusion line. In this regard, the subject technology provides a device that can be used to occlude fluid flow through the infusion pump when the door is open, and which is configured to move relative to the infusion line while the door opens and closes. Moreover, the occluding device is able to automatically enter an occluded configuration when the infusion pump door begins to open in order to prevent inadvertent flow in the infusion line.

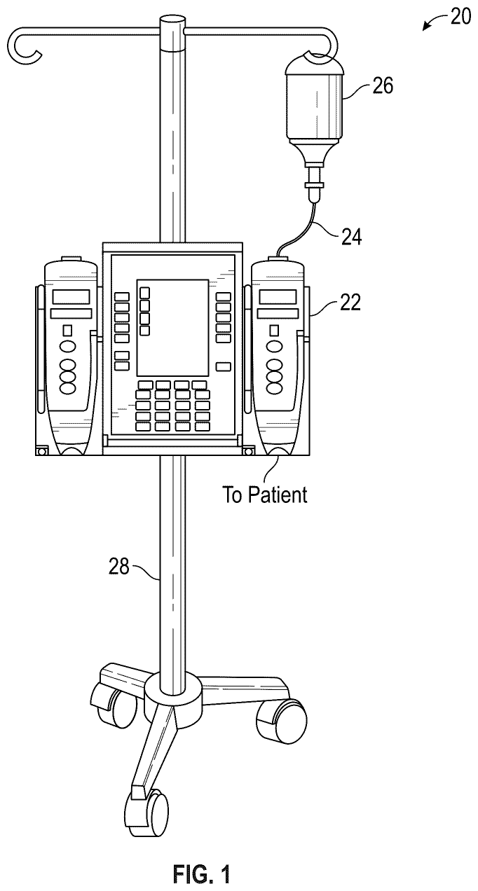

Referring now to the figures, FIG. 1 illustrates an infusion pump loaded with an IV set configured for use, in accordance with some implementations disclosed herein. The IV set 20 includes an IV administration tube 24 in operative engagement with an infusion pump 22. A fluid source 26 can be suspended from appropriate apparatus such as an IV pole 28. The tube 24 is connected between the fluid source 26 and the patient (not shown) so that the patient may receive the fluid of the fluid source at a rate controlled by the infusion pump 22.

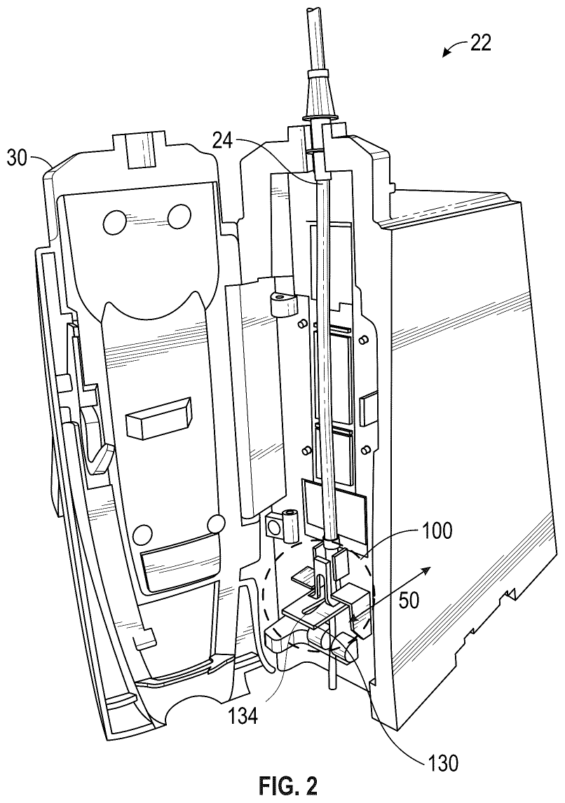

FIG. 2 illustrates a perspective view of the infusion pump shown in FIG. 1 with its door opened, revealing an example IV flow stop coupled thereto, in accordance with some implementations disclosed herein. The infusion pump 22 has a door 30 that is open to expose an interior of the infusion pump 22. As shown, the IV tube 24 is engaged with the infusion pump 22, and the IV flow stop 100 is engaged with the IV tube 24 and the interior of the infusion pump 22. In particular, the IV flow stop 100 is positioned within a recess of a downstream region of the infusion pump 22 and includes a slider 130 that slides in a direction 50 toward or away from the IV tube 24 to occlude or permit fluid flow through the IV tube 24. The IV flow stop 100 protrudes out of the recess such that, when the door 30 of the infusion pump 22 is closed, the door 30 pushes the slider 130 of the IV flow stop 100 further into the recess When the door 30 is opened, a biasing mechanism (not shown) pushes the slider 130 out of the recess into default state. The slider 130 includes an aperture 134 with a varying width that functions as a clamping mechanism that pinches or releases the IV tube 24 to occlude or permit flow through the IV tube 24, depending on the position of the slider 130. Fluid flow is occluded as the slider 130 slides towards the IV tube 24 when the door 30 is closed, and fluid flow is permitted as it slides away from the IV tube 24 when the door 30 is opened.

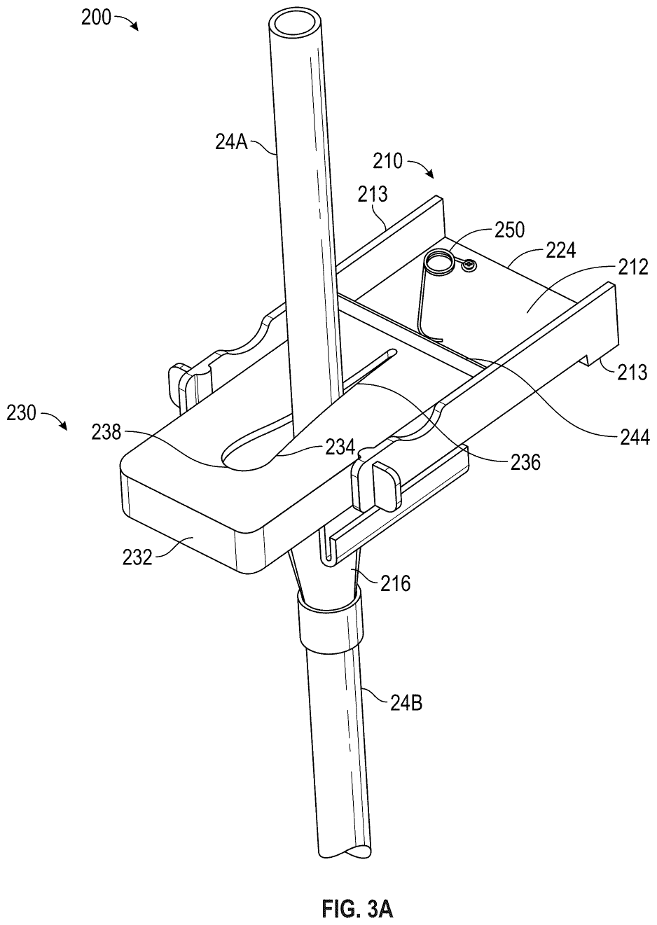

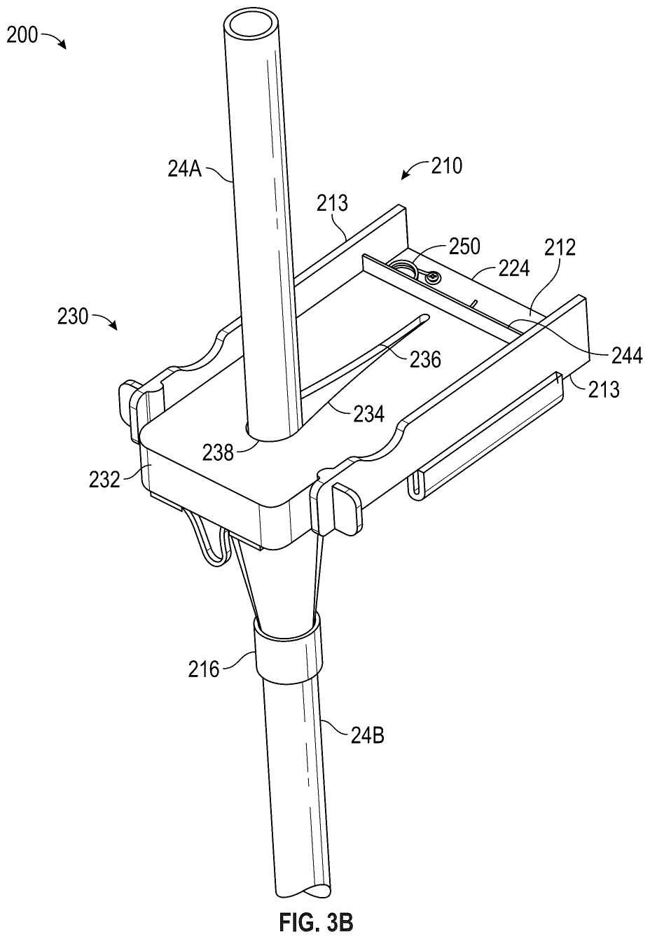

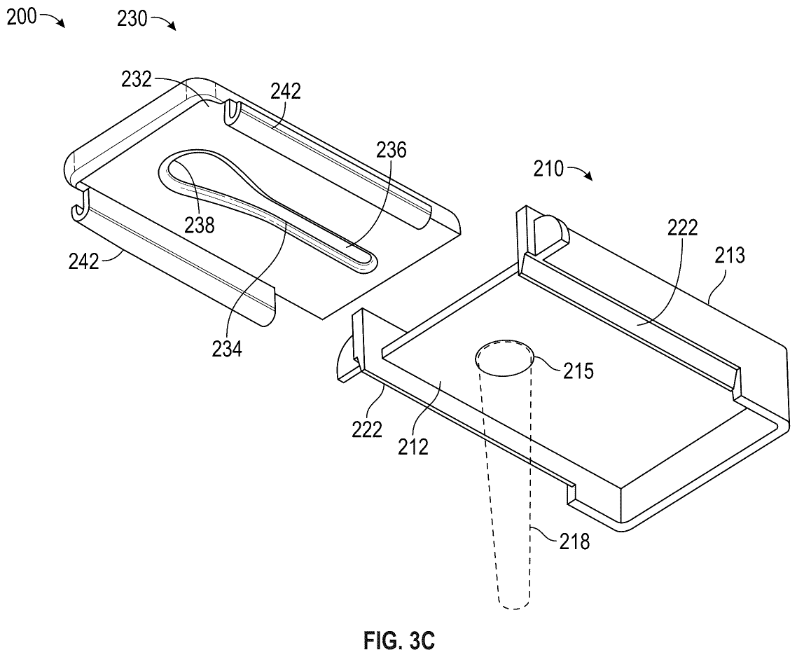

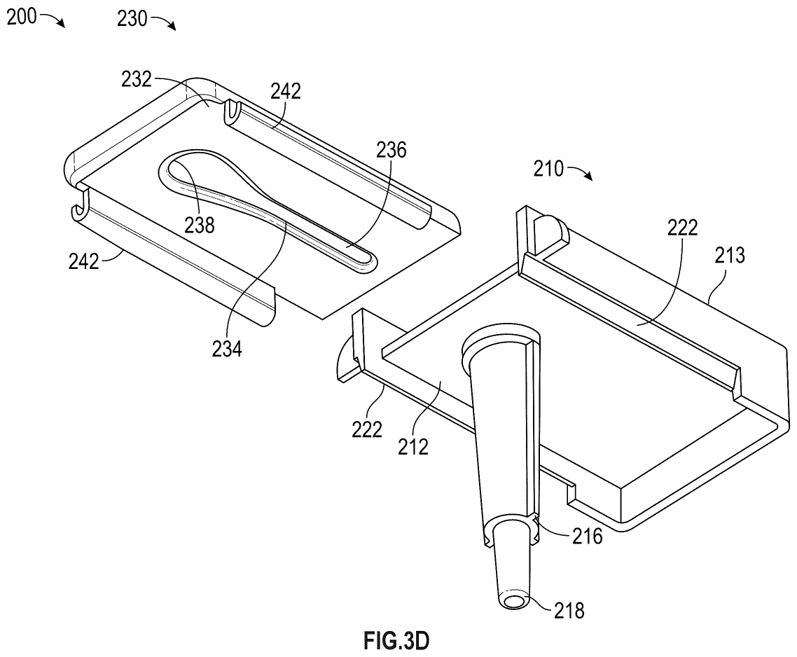

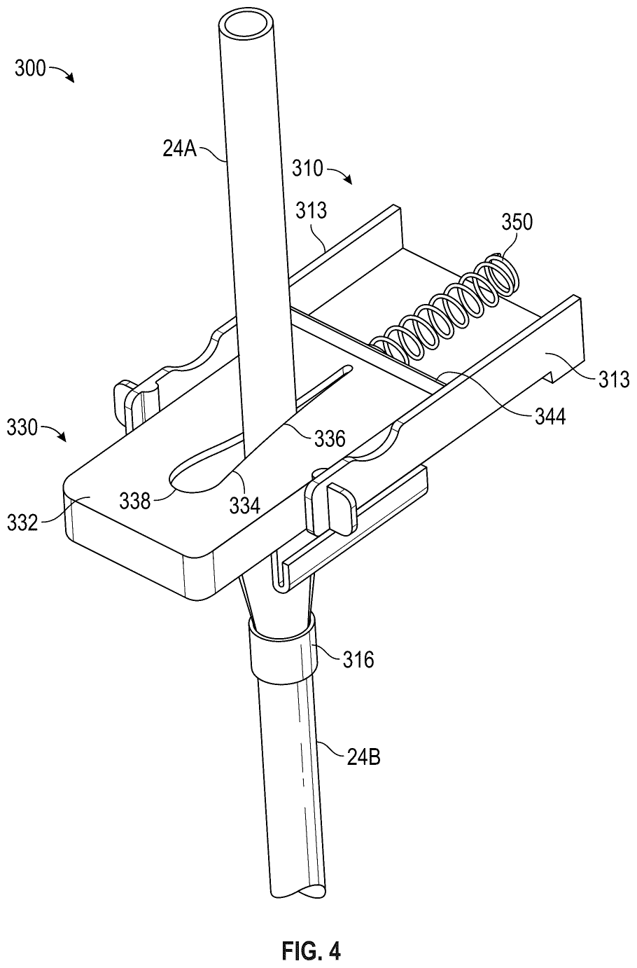

As will be described further, the clamping mechanism 134 includes an occluding region (e.g., the occluding region 236 in FIGS. 3A-3C and the occluding region 336 in FIG. 4) that is configured to pinch the IV tube 24 and a flow region (e.g., the flow region 238 in FIGS. 3A-3C and the flow region 338 in FIG. 4) that is wider than the occlusion region and configured to receive the IV tube 24 without decreasing the diameter of the IV tube 24 (i.e., “release” the IV tube 24). As the slider 130 moves relative to the housing, the clamping mechanism 134 moves relative to the IV tube 24 because the clamping mechanism 134 is part of the slider 130 and the IV tube 24 is coupled to the housing. Consequently, the IV tube 24 slides between the occluding region and the flow region of the clamping mechanism 134 when the slider 130 moves. When the IV tube 24 is in the occluding region, the flow stop 100 is in an occluding configuration, in which flow through the IV tube 24 is occluded. Likewise, when the IV tube 24 is in the flow region, the flow stop 100 is in a flow configuration, in which flow through the IV tube 24 is permitted.

As discussed above, the flow stop 100 is positioned inside a recess in a bottom region (i.e., a downstream region) the infusion pump 22, and the slider 130 of the flow stop 100 protrudes slightly out of the recess such that the door 30 of the infusion pump 22 moves the slider 130 relative to a housing (e.g., the housing 210 illustrated in FIGS. 3A-3D) when the door 30 is opened and closed. The default configuration of the flow stop 100 is the occluded configuration. Thus, when the door 30 is closed (i.e., when the door 30 applies a force to the slider 130), the flow stop 100 is in the flow configuration, and when the door 30 is open (i.e., when the door 30 is not applying a force to the slider 130), the flow stop 100 is in the occluded configuration. Accordingly, in the implementations shown, when the door 30 is closed, the door 30 pushes the slider 130 into the infusion pump 22. As the slider 130 moves inwards relative to the infusion pump 22, the slider 130—and, thus, the clamping mechanism 134—moves relative to the housing such that the flow region of the clamping mechanism 134 moves towards the IV tube 24. When the IV tube 24 is in the flow region, the flow stop 100 is in the flow configuration. When the door 30 begins to open, a biasing member (e.g., the biasing member 250 illustrated in FIGS. 3A-3B or the biasing member 350 illustrated in FIG. 4) causes the slider 130—and thus the clamping mechanism 134—to move outward, such that the occluding region of the clamping mechanism 134 moves towards the IV tube 24. As discussed above, when the IV tube 24 is in the occluding region of the slider 130, the flow stop 100 is in the occluding configuration.

The biasing member ensures that the flow stop 100 immediately and fully enters the occluding configuration upon opening of the door 30. The biasing member can include a spring, a resilient material, etc. that induces, biases, or urges movement of the occluding region of the clamping mechanism 134 (and, thus, the slider 130) towards the IV tube 24 by default. When the door 30 of the infusion pump 22 is closed, the door 30 pushes against the slider 130 (and the biasing member), forcing the slider 130 into the housing and the IV tube 24 into the open region. When the door 30 begins to open and applies less force to the slider 130, the biasing member pushes the slider 130 away from the housing (i.e., towards the door 30) until the IV tube 24 is in the occluding region. In the implementation shown in FIGS. 3A-3B, the biasing member 250 is a torsion spring extending between a distal portion 244 of the slider 230 and a distal portion 224 of the housing 210. The torsion spring is configured to push the slider 230 away from the distal portion 224 of the housing 210 until the IV tube 24 is in the occluding region. In the implementation shown in FIG. 4, the biasing member 350 is a compression spring extending from the distal portion 344 of the slider 330. The compression spring is configured to push the slider 330 away from a back wall of the infusion pump 22 until the IV tube 24 is in the occluding region. Both of these implementations are discussed in greater detail below. In other implementations, the biasing member is a resilient material (e.g., a compressible foam or rubber) that is coupled to the slider 130 such that the resilient material extends in between the flow stop 100 and the back wall of the infusion pump 22. When the door 30 of the infusion pump 22 pushes the slider 130 towards the back wall of the infusion pump 22, the resilient material becomes compressed. When the door 30 is open, the resilient material pushes the slider 130 away from the back wall of the infusion pump 22 until the IV tube 24 is in the occluding region because the resilient material is biased towards its expanded configuration.

FIG. 3A illustrates a perspective top view of a first implementation of the IV flow stop in the occluding configuration, in accordance with some implementations disclosed herein. The first implementation of the IV flow stop 200 is coupled to an IV tube 24A, 24B (i.e., IV tube 24 shown in FIGS. 1 and 2) and has the same features and functions as the IV flow stop 100 described above with respect to FIG. 2, except as described below. The flow stop 200 includes a housing 210, a slider 230, and a biasing member 250.

In the implementation shown, the housing 210 includes a panel 212 that serves as the base of the housing 210 and walls 213 on the perimeter of the housing 210 that are transverse to the panel 212. The housing 210 also includes an opening 215 (shown in FIG. 3C) in the panel 212. This opening is configured to receive the IV tube 24. In particular, the IV tube 24 is transverse to the panel 212 and extends through the housing 210 via the opening. In some implementations, the opening may form a port 216 that extends from the opening transverse to the panel 212 to support the IV tube 24. The port 216 is described in greater detail below with respect to FIG. 3C.

In some implementations, the flow stop 200 is configured to connect two IV tubes 24A and 24B. For example, a first IV tube 24A can connect to a top side (i.e., the upstream side of the flow stop 200 depicted in FIGS. 3A-3B) of the flow stop 200 via the slider 230 (discussed in greater detail below), and a second IV tube 24B can connect to a bottom side (i.e., the downstream side of the flow stop 200 depicted in FIGS. 3C-3D) of the flow stop 200 via the opening 215 or the port 216 on the housing 210.

FIG. 3A also depicts the slider 230 coupled to the housing 210. As shown, the base 232 of the slider 230 is parallel to and supported by the panel 212 of the housing 210, such that slider 230 may slide along a top surface of the panel 212. According to various implementations, the base 232 of the slider 230 is sized and shaped to fit within the walls 213 of the housing 210. The base 232 of the slider 230 includes a clamping mechanism in the form of a clamping aperture 234 that is configured to receive the IV tube 24. The clamping aperture 234 extends through the base 232 and may have a tapered or teardrop shape (as shown in the depicted example). The clamping aperture 234 includes two regions on opposite sides of the teardrop shape: the occluding region 236 and the flow region 238. The tapered shape of the clamping aperture 234 allows the IV tube 24 to smoothly move between the narrow occluding region 236 and the wide flow region 238 by limiting the occurrence of snagging.

In FIG. 3A, the IV tube 24 is coupled to the occluding region 236. In the implementations shown, the occluding region 236 is a slot (and can be referred to as an occlusion slot 236) with a width that is sized to pinch the IV tube 24 shut, thereby occluding flow through the IV tube 24. When the occluding region 236 is aligned with the opening in the housing 210, the flow stop 200 is in the occluding configuration, and flow through the IV tube 24 is occluded. On the other hand, the flow region 238 is an opening (and can be referred to as a flow opening 238) with a diameter that is the same as the same as the outer diameter of the IV tube 24. Accordingly, flow through the IV tube 24 is not obstructed when the IV tube 24 is in the flow region 238. When the flow region 238 is aligned with the opening in the housing 210, the flow stop 200 is in the flow configuration, and flow through the IV tube 24 is permitted.

Finally, FIG. 3A illustrates the biasing member 250 of the flow stop 200. The biasing member 250 is coupled to the housing 210 and the slider 230. In the implementations illustrated in FIG. 3A, the biasing member 250 has a first, distal end and a second, proximal end, where the first end is coupled to a distal portion 224 of the housing 210 and the second end is coupled to a distal portion 244 of the slider 230. The biasing member 250 is a resilient member that biases movement of the slider 230 proximally to align the occluding region 236 with the opening in the housing 210. That is, the biasing member 250 induces the flow stop 200 to enter the occluding configuration by default. The biasing member 250 can be a spring, a mechanism made of a resilient material (e.g., foam or rubber), or any mechanism that otherwise biases the movement of the slider 230 towards the IV tube 24 and, thus, biases the flow stop 200 towards the occluding configuration.

In the implementation shown in FIG. 3A, the biasing member 250 is a torsion spring. Torsion springs are small springs that facilitate a smaller, more compact flow stop 200 while still providing a resistive force that is compatible with the force provided by the door of the infusion pump.

FIG. 3A depicts the first end of the torsion spring 250 fastened to the distal portion 224 of the housing 210 by a screw. Optionally, the first end of the torsion spring 250 can be adhered, bolted, or otherwise secured to the distal portion 224 of the housing. In some implementations, the housing 210 includes a back wall that connects the side walls 213. In those implementations, the first end of the torsion spring 250 can also be coupled to the back wall instead of the distal portion 224 of the housing. FIG. 3A also depicts the second end of the torsion spring 250 coupled to the distal portion 244 of the slider 230. In some implementations, the second end of the torsion spring 250 merely leans against the distal portion 244 of the slider 230, such that when the slider 230 slides relative to the housing 210, the second end of the torsion spring 250 slides along the distal portion (or back wall) 244 of the slider 230. In other implementations, the second end of the torsion spring 250 is secured to the distal portion 244 of the slider 230 using one of the securement methods described above with respect to the first end of the torsion spring 250 (i.e., screw, adhesive, etc.).

In other implementations, the biasing member 250 is a compression spring with a first end coupled to the distal portion 224 of the housing 210 and a second end coupled to the distal portion 244 of the slider 230. The longitudinal axis of the compression spring is parallel to the direction of the sliding motion of the slider 230. A compression spring may be desirable in situations where near-instantaneous entry into the occluding configuration is required because, while compression springs generally occupy more space than torsion springs, compression springs also tend to provide greater resistance than torsion springs.

The biasing member 250 is configured to rest in a “pretensioned,” “expanded,” or “unloaded” position by default. When a force is applied to one or both ends of the biasing member 250 to draw the two ends together, the biasing member 250 is in a “compressed” or “loaded” position. In FIG. 3A, the biasing member 250 is in an unloaded position and separates the distal portion 224 of the housing 210 from the distal portion 244 of the slider 230 by pushing the distal portion 244 proximally and away from the distal portion 224. When the distal portion 224 of the housing 210 is separated from the distal portion 244 of the slider 230, the occluding region/slot 236 is aligned with the opening in the housing 210. Thus, when the biasing member 250 is in the unloaded position, the flow stop 200 is in the occluding configuration. This also means that when there is no outside force pushing the distal portion 244 of the slider 230 towards the distal portion 224 the housing 210 (i.e., the door 30 in FIG. 2 pushing the slider into the infusion pump 22), the biasing member 250 is unloaded and induces the flow stop 200 to enter the occluding configuration. The flow configuration is illustrated in FIG. 3B.

FIG. 3B illustrates a perspective top view of the first implementation of the IV flow stop in the flow configuration, in accordance with some implementations disclosed herein. In FIG. 3B, the biasing member 250 is in the loaded position, and the flow stop 200 is in the flow configuration. When the biasing member 250 is in the loaded position (e.g., in response to the door 30 in FIG. 2 pushing the slider into the infusion pump 22), the distal portion 244 of the slider 230 slides distally towards the distal portion 224 of the housing 210. When the slider 230 slides in this direction, the flow region/opening 238 of the clamping aperture 234 overlaps the opening in the housing 210 and couples to the IV tube 24. Consequently, the flow stop 200 is in the flow configuration.

Because the biasing member 250 is biased towards the unloaded position, when the outside force (e.g., from the door 30 in FIG. 2) is removed from the flow stop 200, the biasing member 250 immediately enters the unloaded configuration. As a result, the distal portion 244 of the slider 230 is pushed away from the distal portion 224 of the housing 210, and the occluding region 236 is pushed towards the opening of the housing 210. Therefore, the biasing member 250 facilitates immediate transition and full entry of the flow stop 200 into the occluding configuration.

FIG. 3C illustrates a perspective bottom view of the first implementation of the IV flow stop with the housing and the slider separated, in accordance with some implementations disclosed herein. FIG. 3C provides an unobstructed view of the opening 215 in the panel 212 of the housing 210. An IV tube can be fed through the opening 215 to couple the IV tube to the housing 210. In some implementations, a tube connector 218 can extend from the opening 215, and an IV tube can couple to the housing 210 via the tube connector 218. The tube connector 218 is particularly desirable in implementations where the flow stop 200 is configured to receive and fluidly connect two separate IV tubes instead of receiving one continuous IV tube.

FIG. 3C also depicts the rails 222 on the housing 210 and the channels 242 on the slider 230. The rails 222 are transverse to the panel 212 of the housing 210 and extend from the walls 213 of the housing 210. The rails 222 are configured to slide inside the channels 242 on the slider 230. As shown, the channels 242 protrude from the sides of the slider 230 and are spaced such that both channels 242 can couple with both rails 222 simultaneously.

FIG. 3D illustrates the IV flow stop of FIG. 3C with a port attached to the opening, in accordance with some implementations disclosed herein. As described above, some implementations of the housing 210 include a port 216 that extends from the opening 215 in the panel 212. FIG. 3D provides an unobstructed view of the port 216 that extends from the panel 212 of the housing 210. As shown, the port 216 protrudes transversely from the panel 212. The port 216 is configured to support and guide the IV tube as the IV tube exits the flow stop 200. In some implementations, the port 216 also includes a tube connector 218, which can receive an IV tube in configurations of the flow stop 200 that are configured to receive two IV tubes (i.e., the tube connector 218 can receive the IV tube 24B in FIG. 3A).

In some implementations, the slider 230 also includes a port that is configured to receive the IV tube 24. In these implementations, the flow stop 200 is in the flow configuration when the port on the slider 230 is fluidly connected to the port 216 on the housing 210. Likewise, the flow stop 200 is in the occluding configuration when the port on the slider 230 is not aligned with the port 216 on the housing 210. Optionally, the port on the slider 230 receives a first IV tube 24A, and the port 216 on the housing 210 receives a second IV tube 24B.

FIG. 4 illustrates a perspective top view of a second implementation of the IV flow stop in the occluding configuration, in accordance with some implementations disclosed herein. The second implementation of the IV flow stop 300 is coupled to an IV tube 24A, 24B (i.e., IV tube 24 shown in FIGS. 1 and 2) and has the same features and functions as the IV flow stops 100 and 200 described above, except as described below. The flow stop 300 includes a housing 310, a slider 330, and a biasing member 350.

The housing 310 includes side walls 313 that are transverse to the base 312 of the housing 310 and transverse to the direction of movement of the slider 330. The housing 310 also includes an opening in the base 312 that is configured to receive the IV tube 24. Optionally, the housing 310 also includes a port 316 (akin to the port 216 in the flow stop 200) that extends from the opening to support and guide the IV tube 24. Optionally, the flow stop 300 is configured to connect to IV tubes 24A and 24B, which is discussed in greater detail above with respect to FIG. 3A.

FIG. 4 also depicts the slider 330 coupled to the housing 310. As shown, the base 332 of the slider 330 is transverse to a back wall 344 of the slider 330, and the slider 330 is sized and shaped to fit within the walls 313 of the housing 310 when the slider 330 slides onto the housing 310. Furthermore, the base 332 of the slider 330 includes a clamping aperture 334 that is configured to receive the IV tube 24. The clamping aperture 334 is the same as the clamping aperture 234 described above with respect to the first implementation flow stop 200. Accordingly, the clamping aperture 334 has an occluding region or slot 336 and a flow region or opening 338 and can slide relative to the opening in the housing 310 to transition the flow stop 300 between the occluding configuration and the flow configuration. The clamping aperture 234 tapers to transition between the occluding region 336 and the flow region 338, which allows the IV tube 24 to smoothly move between the occluding region 236 and the flow region 238 with limited catching or snagging.

In some implementations of the flow stop 300, like the slider 230 in the flow stop 200, the slider 330 also includes a port that is configured to receive the IV tube 24.

Finally, FIG. 4 illustrates the biasing member 350 of the flow stop 300. The biasing member 350 is coupled to the back wall 344 of the slider 330. Like the biasing member 250 discussed above with respect to FIGS. 3A-3C, the biasing member 350 can be any type of spring or a compressible mechanism that is resiliently biased towards a default, uncompressed (i.e., expanded) configuration. In the implementation shown in FIG. 4, the biasing member 350 is a compression spring with a longitudinal axis that is transverse to the back wall 344. The compression spring 350 protrudes off the back wall 344 of the slider 330 and extends beyond a distal end of the housing 310 such that the compression spring contacts an interior of an infusion pump (e.g., infusion pump 22 in FIGS. 1 and 2). The compression spring 350 has a default position in which it is unloaded or expanded, shown in FIG. 4, and an unloaded or compressed configuration. The compression spring 350 is in the unloaded position when there are no external forces pushing the slider 330 into the infusion pump (i.e., the door 30 in FIG. 2 is open). When the compression spring 350 is in the unloaded position, the compression spring 350 pushes the slider 330 proximally (i.e., out of the infusion pump 22 in FIGS. 1 and 2) such that the occlusion region/slot 336 of the clamping aperture 334 aligns with the opening in the housing 310. That is, when the compression spring 350 is expanded, the flow stop 300 is in the occluded configuration.

On the other hand, when a force pushes the slider 330 towards the back wall of the infusion pump (i.e., when the door 30 in FIG. 2 pushes the slider into the infusion pump 22), the compressing spring 350 enters the unloaded configuration, and the flow region/opening 338 of the clamping aperture 334 aligns with the opening in the housing 310. Accordingly, the flow stop 300 enters the flow configuration.

Because the compression spring 350 is biased towards the unloaded configuration, when the outside force (e.g., the force from the door 30 in FIG. 2) is removed from the flow stop 300, the compression spring 350 immediately returns to the unloaded configuration. Consequently, the flow stop 300 immediately returns to the occluding configuration. This means that the compression spring 350 facilitates immediate transition and full entry of the flow stop 300 into the occluding configuration.

The IV flow stops disclosed herein are compatible with other accessories of the IV set. For example, the IV flow stops can be coupled to a flow stop detector and a processor. The flow stop detector is coupled to the IV tube or the infusion pump and configured to identify the configuration of the IV flow stop (i.e., whether the flow stop is in the occluding or flow configuration). The flow stop detector is also configured to transmit a signal to the processor, which provides an alert signal to caregivers or clinicians that are monitoring the IV set when the flow stop changes configurations.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.
Clause 1. An intravenous (IV) flow stop having an occluding configuration and a flow configuration, the flow stop comprising: a housing comprising an opening configured to receive an IV tube; a slider slidably coupled to the housing to transition the flow stop between the occluding configuration and the flow configuration, wherein the slider comprises a clamping aperture that has an occlusion slot, has a flow opening, and is configured to receive the IV tube; and a biasing member coupled to the slider and the housing to bias movement of the slider along the housing, such that the flow stop is biased towards the occluding configuration, wherein, when the flow stop is in the occluding configuration, the occlusion slot overlaps the opening in the housing to occlude the IV tube, wherein, when the flow stop is in the flow configuration, the flow opening overlaps the opening in the housing to permit flow through the IV tube.
Clause 2. The flow stop of Clause 1, wherein the flow stop is configured to be coupled to an interior of an infusion pump.
Clause 3. The flow stop of Clause 2, wherein the IV flow stop is configured to enter the flow configuration when a door of the infusion pump is closed because the closed door slides the slider until the flow opening overlaps the opening in the housing.
Clause 4. The flow stop of Clause 2, wherein the IV flow stop is configured to enter the occluding configuration automatically when a door of the infusion pump is begins to open because the biasing member slides the slider until the occlusion slot overlaps the opening in the housing.
Clause 5. The flow stop of Clause 1, wherein the IV tube is transverse to the housing and the slider slides parallel to the housing.
Clause 6. The flow stop of Clause 1, further comprising a port extending from the opening of the housing transverse to the housing, wherein the port is configured to receive the IV tube.
Clause 7. The flow stop of Clause 1, wherein the occlusion slot is narrower than the flow opening, such that the occlusion slot is configured to pinch the IV tube to occlude the IV tube.
Clause 8. The flow stop of Clause 1, wherein the biasing member comprises a torsion spring.
Clause 9. The flow stop of Clause 8, wherein a first end of the torsion spring is coupled to a distal portion of the housing and a second end of the torsion spring is coupled to a distal portion of the slider.
Clause 10. The flow stop of Clause 9, wherein, in a pretensioned position, the torsion spring is configured to urge the distal portion of the slider away from the distal portion of the housing, and, in a loaded position, the torsion spring allows the distal portion of the housing and the distal portion of the slider to slide towards each other.
Clause 11. The flow stop of Clause 10, wherein the flow stop is in the occluding configuration when the torsion spring is in the pretensioned position, and the flow stop is in the flow configuration when the torsion spring is in the loaded position.
Clause 12. An intravenous (IV) flow stop having an occluding configuration and a flow configuration, the flow stop comprising: a housing configured to couple with an IV set; a slider slidably coupled to the housing and comprising a clamping mechanism that is configured to receive the IV set and slide relative to the IV set when the slider slides relative to the housing; and a biasing member coupled to the slider and the housing, wherein the biasing member biases the flow stop towards the occluding configuration, wherein the clamping mechanism occludes flow through the IV set when the flow stop is in the occluding configuration and permits flow through the IV set when the flow stop is in the flow configuration.
Clause 13. The flow stop of Clause 12, wherein the clamping mechanism comprises: an occluding region configured to occlude the IV set by engaging the IV set when the flow stop is in the occluding configuration; and a flow region configured to permit flow through the IV set by engaging the IV set when the flow stop is in the flow configuration.
Clause 14. The flow stop of Clause 12, wherein the biasing member comprises a torsion spring.
Clause 15. The flow stop of Clause 14, wherein the flow stop is in the occluding configuration when the torsion spring is in a pretensioned position, and the flow stop is in the flow configuration when the torsion spring is in a loaded position.
Clause 16. The flow stop of Clause 12, wherein the biasing member comprises a compression spring.
Clause 17. An intravenous (IV) flow stop having an occluding configuration and a flow configuration, the flow stop comprising: a housing configured to couple with an IV set; a slider slidably coupled to the housing, comprising a clamping mechanism, and configured to couple with the IV set; and a biasing member protruding from a distal portion of the slider, wherein the biasing member is configured to bias the flow stop towards the occluding configuration, wherein, to enter the occluding configuration, the biasing member is configured to expand to slide the slider proximally relative to the housing, such that the clamping mechanism occludes flow through the IV set, wherein, to enter the flow configuration, the biasing member is configured to contract to slide the housing distally relative to the slider, such that the clamping mechanism permits flow through the IV set.
Clause 18. The flow stop of Clause 17, wherein the housing comprises a port configured to connect the housing to the IV set.
Clause 19. The flow stop of Clause 17, wherein the clamping mechanism comprises an occluding region configured to occlude the IV set and a flow region configured to permit flow through the IV set, wherein the clamping mechanism is configured to move relative to the IV set when the slider slides relative to the housing, such that the occluding region is configured to couple with the IV set when the flow stop is in the occluding configuration and the flow region is configured to couple with the IV set when the flow stop is in the flow configuration.
Clause 20. The flow stop of Clause 17, wherein the biasing member comprises a compression spring.
Clause 21. An intravenous (IV) delivery system comprising an infusion pump and an IV flow stop. The infusion pump comprises an inlet configured to receive an IV tube, an outlet configured to receive the IV tube, a recess positioned in between the inlet and an outlet, and a door configured to open and close. The IV flow stop is positioned inside the recess and transitions between an occluding configuration and a flow configuration. The flow stop comprises a housing, a slider that protrudes slightly out of the recess and is slidably coupled to the housing to transition the flow stop between the occluding configuration and the flow configuration, and a biasing member coupled to the slider and the housing to bias the flow stop towards the occluding configuration. The housing comprises an opening configured to receive the IV tube. The slider comprises a clamping aperture that has an occlusion slot, has a flow opening, and is configured to receive the IV tube. When the flow stop is in the occluding configuration, the occlusion slot overlaps the opening in the housing to occlude the IV tube, and when the flow stop is in the flow configuration, the flow opening overlaps the opening in the housing to permit flow through the IV tube.

### Further Considerations

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) flow stop having an occluding configuration and a flow configuration, the flow stop comprising:
a housing comprising an opening configured to receive an IV tube;
a slider slidably coupled to the housing to transition the flow stop between the occluding configuration and the flow configuration, wherein the slider comprises a clamping aperture that has an occlusion slot, has a flow opening, and is configured to receive the IV tube; and
a biasing member coupled to the slider and the housing to bias movement of the slider along the housing, such that the flow stop is biased towards the occluding configuration,
wherein, when the flow stop is in the occluding configuration, the occlusion slot overlaps the opening in the housing to occlude the IV tube,
wherein, when the flow stop is in the flow configuration, the flow opening overlaps the opening in the housing to permit flow through the IV tube.

2. The flow stop of claim 1, wherein the flow stop is configured to be positioned in a recess of an infusion pump with the slider partially protruding out of the recess, such that the slider is configured to receive a force from a door of the infusion pump and slide further into the recess when the door is closed.

3. The flow stop of claim 2, wherein the flow stop is configured to enter the flow configuration when the door is closed because the force from the door slides the slider further into the recess until the flow opening overlaps the opening in the housing.

4. The flow stop of claim 2, wherein the flow stop is configured to enter the occluding configuration when the door begins to open because the biasing member slides the slider out of the recess until the occlusion slot overlaps the opening in the housing.

5. The flow stop of claim 2, wherein the flow stop can only enter the flow configuration when the force from the door that slides the slider further into the recess exceeds a force from the biasing member that slides the slider out of the recess.

6. The flow stop of claim 1, wherein the IV tube is transverse to the housing, and the slider slides parallel to the housing.

7. The flow stop of claim 1, further comprising a port extending from the opening of the housing transverse to the housing, wherein the port is configured to receive the IV tube.

8. The flow stop of claim 1, wherein the occlusion slot is narrower than the flow opening, such that the occlusion slot is configured to pinch the IV tube to occlude the IV tube.

9. The flow stop of claim 1, wherein the biasing member comprises a torsion spring.

10. The flow stop of claim 1, wherein a first end of the biasing member is coupled to a distal portion of the housing and a second end of the biasing member is coupled to a distal portion of the slider, such that the biasing member is configured to:
in an unloaded position, urge the distal portion of the slider away from the distal portion of the housing; and
in a loaded position, allow the distal portion of the housing and the distal portion of the slider to slide towards each other.

11. The flow stop of claim 9, wherein the flow stop is in the occluding configuration when the biasing member is in the unloaded position, and the flow stop is in the flow configuration when the biasing member is in the loaded position.

12. An intravenous (IV) flow stop having an occluding configuration and a flow configuration, the flow stop comprising:
a housing configured to couple with an IV set;
a slider slidably coupled to the housing and comprising a clamping mechanism that is configured to receive the IV set and slide relative to the IV set when the slider slides relative to the housing; and
a biasing member coupled to the slider and the housing, wherein the biasing member biases the flow stop towards the occluding configuration,
wherein the clamping mechanism occludes flow through the IV set when the flow stop is in the occluding configuration and permits flow through the IV set when the flow stop is in the flow configuration.

13. The flow stop of claim 12, wherein the clamping mechanism comprises:
an occluding region configured to occlude the IV set by engaging the IV set when the flow stop is in the occluding configuration; and
a flow region configured to permit flow through the IV set by engaging the IV set when the flow stop is in the flow configuration.

14. The flow stop of claim 12, wherein the biasing member comprises a torsion spring.

15. The flow stop of claim 14, wherein the flow stop is in the occluding configuration when the torsion spring is in a pretensioned position, and the flow stop is in the flow configuration when the torsion spring is in a loaded position.

16. The flow stop of claim 12, wherein the biasing member comprises a compression spring.

17. An intravenous (IV) flow stop having an occluding configuration and a flow configuration, the flow stop comprising:
a housing configured to couple with an IV set;
a slider slidably coupled to the housing, comprising a clamping mechanism, and configured to couple with the IV set; and
a biasing member protruding from a distal portion of the slider, wherein the biasing member is configured to bias the flow stop towards the occluding configuration,
wherein, to enter the occluding configuration, the biasing member is configured to expand to slide the slider proximally relative to the housing, such that the clamping mechanism occludes flow through the IV set,
wherein, to enter the flow configuration, the biasing member is configured to contract to slide the housing distally relative to the slider, such that the clamping mechanism permits flow through the IV set.

18. The flow stop of claim 17, wherein the housing comprises a port configured to connect the housing to the IV set.

19. The flow stop of claim 17,
wherein the clamping mechanism comprises an occluding region configured to occlude the IV set and a flow region configured to permit flow through the IV set,
wherein the clamping mechanism is configured to move relative to the IV set when the slider slides relative to the housing, such that the occluding region is configured to couple with the IV set when the flow stop is in the occluding configuration and the flow region is configured to couple with the IV set when the flow stop is in the flow configuration.

20. The flow stop of claim 17, wherein the biasing member comprises a compression spring.
